(19) United States
(12) Patent Application Publication
(10) Pub. No.: US 2024/0083937 A1
(43) Pub. Date: Mar. 14, 2024

(54) TRIPLE C-G-T BASE CODED NUCLEOBASE AMINO ACID ITS SYNTHESIS AND PEPTIDE FORMATION

(71) Applicant: COUNCIL OF SCIENTIFIC AND INDUSTRIAL RESEARCH, New Delhi (IN)

(72) Inventors: Mahendra Ashok Wagh, Maharashtra (IN); Gangadhar Jessy Sanjayan, Maharashtra (IN)

(21) Appl. No.: 17/961,220

(22) Filed: Oct. 6, 2022

(30) Foreign Application Priority Data
Aug. 22, 2022 (IN) 202211047552

(51) Int. Cl. C07K 1/02; C07K 1/06

(52) U.S. Cl. CPC C07K 1/02; C07K 1/06

(57) ABSTRACT

Triple G-C-T base coded nucleobase amino acids according to Formula (I):

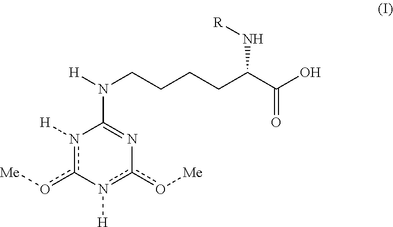

wherein R is —H or -Boc, are provided as building blocks for peptide sequences. The compound of formula (I) includes three recognition sites, DDA (G mimic), DAA (C mimic) and ADA (T mimic) that can simultaneously interact with two sets of nucleobases (C-A or G-A), at any given time. A one-step synthetic process for the triple G-C-T base coded nucleobase amino acids is provided.

### CROSS-REFERENCES TO RELATED APPLICATIONS

This application claims benefit of priority under 35 U.S.C. § 119(a)-(d) to Indian Patent Application No. 202211047552, filed Aug. 22, 2022, which is incorporated by reference herein in its entirety.

### TECHNICAL FIELD

The present invention relates to a triple G-C-T base coded nucleobase amino acid, process for the preparation and application in peptide formation thereof. More particularly, the present invention relates to a triple G-C-T base coded nucleobase amino acid of Formula (I) as building blocks for peptide sequences and its one step synthetic process thereof.

### BACKGROUND

Nucleobase amino acids (NBA), as a distinctive class of unnatural amino acids have ushered into prominence primarily owing to their utility in nucleic acid recognition and nucleic acid-protein interaction studies. These amino acids, typically bearing synthetic or native nucleobases on the side chain of α-amino acid residues, are being increasingly employed for designing of polypeptides and proteins to interact with their nucleic acid substrates using Watson-Crick and other base pairing interactions.

Several Nucleobase amino acids have been reported recently; among them the prominent ones are alanyl-based NBAs (1-4 shown below) synthesized from serine lactone following multi-step synthetic routes. The homologs of alanyl NBA such as homoalanyl 5, 6 and norvalyl 7, 8 NBAs also have been explored for their utility in studying nucleic acid-protein recognitions.

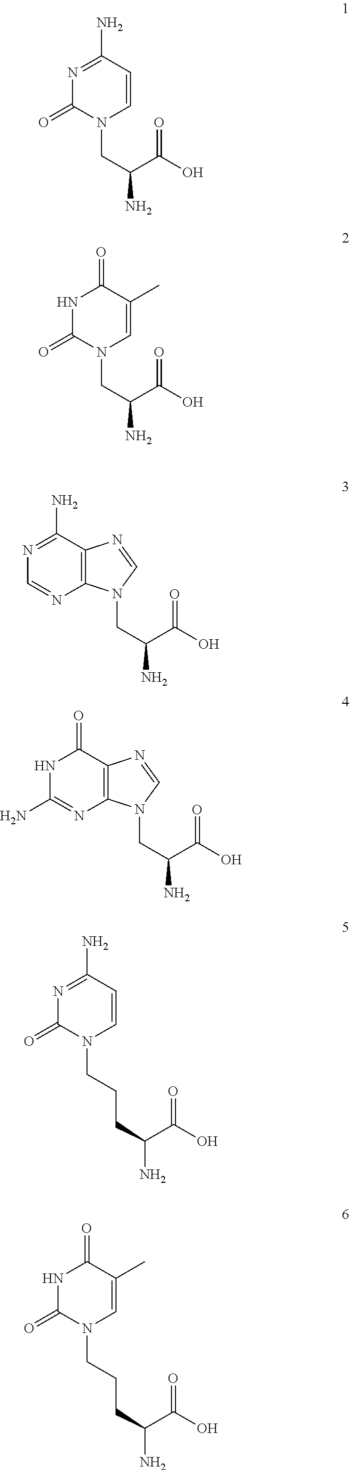 [SMILES: Nc1ccn(C[C@H](N)C(=O)O)c(=O)n1 ; Cc1cn(C[C@H](N)C(=O)O)c(=O)[nH]c1=O ; Nc1ncnc2c1ncn2C[C@H](N)C(=O)O ; Nc1nc2c(ncn2C[C@H](N)C(=O)O)c(=O)[nH]1 ; Nc1ccn(CCC[C@H](N)C(=O)O)c(=O)n1 ; Cc1cn(CCC[C@H](N)C(=O)O)c(=O)[nH]c1=O]

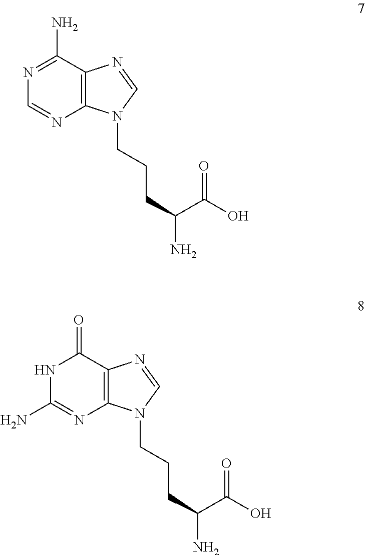 [SMILES: Nc1ncnc2c1ncn2CCC[C@H](N)C(=O)O ; Nc1nc2c(ncn2CCC[C@H](N)C(=O)O)c(=O)[nH]1]

Recently, efforts are being made to develop NBA-based polypeptides and peptide nucleic acids (PNAs) containing double-sided Janus bases, which can code for two nucleobases simultaneously which are thus capable of bifacial recognition. The twin binding faces of Janus nucleobases facilitate their binding to both of the complementary strands of target RNA or DNA with stronger affinity.

### SUMMARY

With a need for further development, the present inventors pursued their research to provide a triple G-C-T nucleobase amino acid (G-C-T_{NBA}), featuring three recognition sites that can simultaneously interact with two sets of nucleobases (C-A or G-A), at any given time.

A main objective of the present disclosure is to provide a triple G-C-T base coded nucleobase amino acid.

Another objective of the present disclosure is to provide a one-step synthetic process for the preparation of a triple G-C-T base coded nucleobase amino acid.

Yet another objective of the present disclosure is to provide an application of a triple G-C-T base coded nucleobase amino acid in the formation of peptide sequences.

Accordingly, to accomplish the objectives, the present disclosure provides a triple G-C-T base coded nucleobase amino acid of Formula (I):

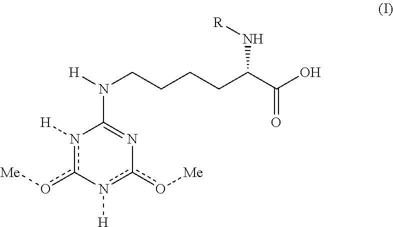

In formula (I), R is H or -Boc. The compound of formula (I) includes three recognition sites, DDA (G mimic), DAA (C mimic) and ADA (T mimic) that can simultaneously interact with two sets of nucleobases (C-A or G-A), at any given time.

In embodiments, a triple G-C-T base coded nucleobase amino acid of Formula (9) is provided, wherein the free triple G-C-T amino acid of Formula (9) can exist in the form (9′) owing to the prototropy effect leading to “G-C” inversion. The compound of formula (9) displays the G and T faces and the compound of Formula (9′) display C and T faces as shown in FIG. 1 suggesting that it can simultaneously interact with two sets of nucleobases (C-A or G-A), at any given time.

The triple G-C-T (guanine-cytosine-thymine) nucleobase amino acid (NBA) includes three recognition faces, DDA (G mimic), DAA (C mimic) and ADA (T mimic), that are complementary to native nucleobases cytosine (C), guanine (G), and adenine (A), respectively.

Another embodiment provides a one-step synthetic process for the preparation of free triple G-C-T amino acid of Formula (9). The process includes:
(a) reacting 2-chloro-4,6-dimethoxy-1,3,5-triazine (10) with α-Boc lysine in the presence of base and solvent at a temperature in the range of 35-40° C. for 1 hour to 2 hours to obtain Boc-protected G-C-T-amino acid intermediate (11); and
(b) deprotecting Boc-group and demethylating intermediate (11) by using HBr/AcOH at a temperature from 35° C. to 40° C. for 1 hour to obtain free G-C-T amino acid of formula (9) in quantitative yield.

In embodiments, a single step process is provided for synthesis of the Boc-protected G-C-T-amino acid (11) key intermediate:

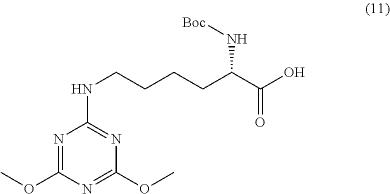 [SMILES: COc1nc(NCCCC[C@H](NC(=O)OC(C)(C)C)C(=O)O)nc(OC)n1]

The process includes reacting 2-chloro-4,6-dimethoxy-1,3,5-triazine (10) with α-Boc lysine in the presence of base and solvent.

The Boc-protected G-C-T-amino acid (11) acts as a key intermediate to introduce the G-C-T NBA building block into peptides sequences from both the N-terminus and the C-terminus.

Yet another embodiment provides a process for the synthesis of peptide sequences/assembly at the backbone of nucleobase, wherein said process comprises:
(i) coupling the C-termini of Boc-protected G-C-T-amino acid (11) with alpha-amino acid to obtain the peptides;
(ii) deprotecting the Boc group to obtain free peptide amine;
(iii) Coupling the free peptide amine at N-terminus further with Boc-protected alpha amino acid to obtain the Boc-protected peptide sequence followed by concurrent saponification, deprotection and O-demethylation to obtain the desired peptide sequence.

Due to availability of both C and N termini for further modifications, the triple G-C-T nucleobase amino acid (9) and (11) finds wide-ranging application for nucleic acid recognition and nucleic acid-peptide/protein interaction studies.

Acronyms used herein:
HBr: Hydrobromic acid
AcOH: Acetic acid
HBTU: Hexafluorophosphate Benzotriazole Tetramethyl Uronium
HOAt: 1-Hydroxy-7-azabenzotriazole
TFA: Trifluoroacetic acid
DCM: Dichloromethane

### BRIEF DESCRIPTION OF THE DRAWINGS

FIG. 1: Depicts Triple G-C-T base coded nucleobase amino acid NBA 9 displaying G and T bases and tautomerism leading to “G-C” inversion to form 9′ displaying C and T bases.

FIG. 2A depicts a molecular structure of the model compound 20 (left), its tautomerized form 20′ (center), and duplex formation 20:20′ (right) owing to G-C canonical Watson-Crick-type hydrogen-bonding, confirming that the present compound could exist in both G (20) and C (20′) forms.

FIG. 2B depicts a crystal structure of a model compound 20 (left) and its dimer showing G-C-type hydrogen bonding (right).

FIG. 3 is a reaction scheme for the synthesis of fully deprotected peptide analogues disclosed herein.

### DETAILED DESCRIPTION

The invention now will be described in detail in connection with certain preferred and optional embodiments, so that various aspects thereof may be more fully understood and appreciated.

Embodiments herein provide a triple G-C-T base coded nucleobase amino acid of Formula (I):

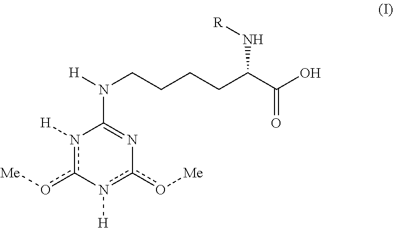

wherein, R=H or -Boc.

The compound of formula (I) includes three recognition sites, DDA (G mimic), DAA (C mimic) and ADA (T mimic) that can simultaneously interact with two sets of nucleobases (C-A or G-A), at any given time.

It should be understood that formula (I) encompasses compounds of formula (Ia) and compounds of formula (Ib):

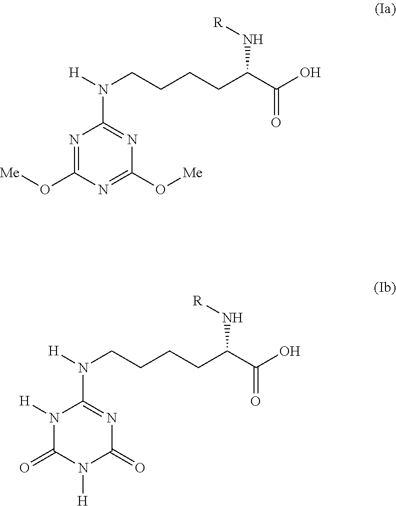

In formula (Ia) and formula (Ib), R is H or -Boc.

In embodiments, a triple G-C-T base coded nucleobase amino acid of Formula (9) is provided, wherein the free triple G-C-T amino acid of Formula (9) can exist in the form (9′) owing to the prototropy effect leading to “G-C” inversion.

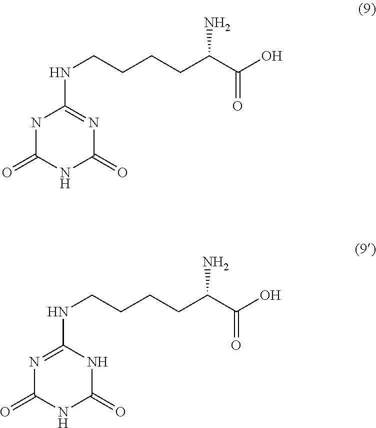 [SMILES: N[C@@H](CCCCNc1nc(=O)[nH]c(=O)[nH]1)C(=O)O ; N[C@@H](CCCCNc1nc(=O)[nH]c(=O)[nH]1)C(=O)O]

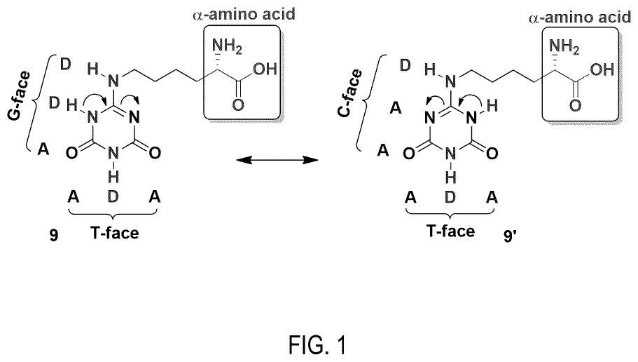

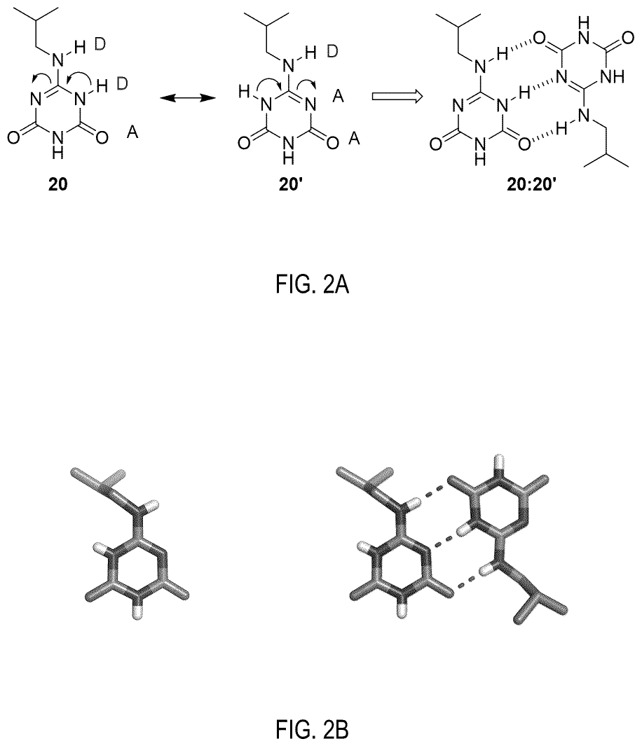

As illustrated in FIG. 1, the compound of formula (9) displays the G and T faces, and the compound of Formula (9′) displays C and T faces, suggesting that it can simultaneously interact with two sets of nucleobases (C-A or G-A), at any given time.

The triple G-C-T (guanine-cytosine-thymine) nucleobase amino acid (NBA) of Formula (9) includes three recognition faces, DDA (G mimic), DAA (C mimic), and ADA (T mimic), that are complementary to native nucleobases cytosine (C), guanine (G), and adenine (A), respectively.

Further embodiments herein provide a one-step synthetic process for the preparation of free triple G-C-T amino acid of Formula (9). The process includes:
(a) reacting 2-chloro-4,6-dimethoxy-1,3,5-triazine (10) with α-Boc lysine in the presence of base and solvent at a temperature from 35° C. to 40° C. for 1 hour to 2 hours to obtain Boc-protected G-C-T-amino acid intermediate (11); and
(b) deprotecting Boc-group and demethylating intermediate (11) by using HBr/AcOH at a temperature from 35° C. to 40° C. for 1 hour to obtain free G-C-T amino acid of formula (9) in quantitative yield.

The base for the reaction is selected from organic bases such as N,N-diisopropylethylamine (DIPEA) and triethylamine (Et₃N). In a particularly useful embodiment, base is N,N-diisopropylethylamine (DIPEA).

The solvent comprises polar protic solvent such as methanol and ethanol. In particularly useful embodiment, solvent is dry methanol.

The process is carried out at ambient temperature, such as from 35° C. to 40° C.

Another embodiment of the present invention provides a Boc-protected triple G-C-T nucleobase amino acid of the Formula (11) as a building block for peptide sequences:

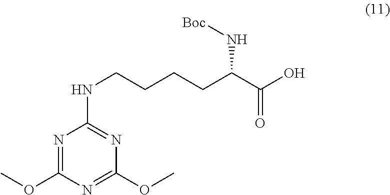 [SMILES: COc1nc(NCCCC[C@H](NC(=O)OC(C)(C)C)C(=O)O)nc(OC)n1]

The Boc-protected triple G-C-T nucleobase amino acid of the Formula (11) includes three recognition sites, DDA (G mimic), DAA (C mimic), and ADA (T mimic), that can simultaneously interact with two sets of nucleobases (C-A or G-A), at any given time.

In embodiments, a single step process is provided for the synthesis of the Boc-protected G-C-T-amino acid (11), a key intermediate, wherein said process comprises of reacting 2-chloro-4,6-dimethoxy-1,3,5-triazine (10) with α-Boc lysine in the presence of base and solvent as depicted below in Scheme 1.

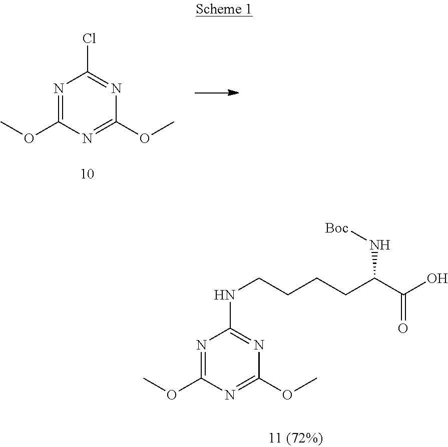

The Boc-protected G-C-T-amino acid (11) acts as a key intermediate to introduce the G-C-T NBA building block into peptides sequences from both the N-terminus and the C-terminus.

The compound (11) is synthesized in multi-gram scale in one step using cheap and commercially available 2-chloro-4,6-dimethoxy-1,3,5-triazine (10). The compound (10) is readily prepared in large amounts starting from cyanuric chloride by a process known in the art, such as the processes disclosed in J. Org. Chem., 2018, 83, 10916-10921 and J. Org. Chem., 2019, 84, 5893-5898.

The base for the reaction is selected from organic bases such as N,N-diisopropylethylamine (DIPEA) and triethylamine (Et₃N). In a particularly useful embodiment, the base is N,N-diisopropylethylamine (DIPEA).

The solvent comprises a polar protic solvent such as methanol and ethanol. In a particularly useful embodiment, solvent is dry methanol.

The process is carried out at ambient temperature, such as from 35° C. to 40° C.

The Boc-protected G-C-T-amino acid of Formula (11) acts as a key NBA building block intermediate, which can be incorporated into peptide sequences from both N-terminus and C-terminus.

Yet another embodiment provides a general process for the synthesis of peptide assembly from Boc-protected G-C-T-amino acid of Formula (11). The process includes:
(i) coupling the C-termini of Boc-protected G-C-T-amino acid (11) with alpha-amino acid in the presence of HBTU/HOAt in DMF at a temperature from 0° C. to 40° C. for 17 hours to 18 hours to obtain the peptides;
(ii) deprotecting the Boc group at a temperature from 0° C. to 40° C. for 30 minutes to 40 minutes by using a TFA:DCM (1:1) mixture to obtain free peptide amine;
(iii) coupling the free peptide amine at the N-terminus further with Boc-protected alpha amino acid in the presence of HBTU/HOAt in DMF at a temperature from 0° C. to 40° C. for 17 hours to 18 hours to obtain the Boc-protected peptide sequence followed by concurrent saponification, deprotection and O-demethylation, to obtain the desired peptide sequence.

In embodiments, a process is provided for the preparation of a tripeptide (15) from Boc-protected G-C-T-amino acid of Formula (11) The process includes:
(i) coupling the C-termini of Boc-protected G-C-T-amino acid (11) with NH₂-Phe-OMe in the presence of HBTU/HOAt in DMF at a temperature from 0° C. to 40° C. for a period of 17 hours to 18 hours to afford the dipeptide (12);
(ii) deprotecting selectively Boc group of dipeptide (12) at a temperature from 0° C. to 40° C. by using TFA:DCM (1:1) mixture for 30 to 40 minutes to obtain free peptide amine (13);
(iii) coupling the free peptide amine (13) at the N-end with Boc-Ala-OH in the presence of HBTU/HOAt in DMF at a temperature from 0° C. to 40° C. to afford the Boc-protected tripeptide (14); and
(iv) subjecting the Boc-protected tripeptide (14) to ester saponification at a temperature from 0° C. to 40° C. in water for 4 hours, followed by concurrent Boc-deprotection and O-demethylation by using HBr in AcOH at a temperature from 35° C. to 40° C. for 1 hour to afford deprotected tripeptide (15).

In embodiments, a process is provided for preparing pentapeptide (19) from Boc-protected G-C-T-amino acid of Formula (11). The process comprises:
(i) coupling the C-termini of Boc-protected G-C-T-amino acid (11) with NH₂-Val-Phe-OMe in the presence of HBTU/HOAt in DMF at a temperature from 0° C. to 40° C. for 17 to 18 hours to afford the tripeptide (16);
(ii) deprotecting selectively Boc group of tripeptide (16) at a temperature from 0° C. to 40° C. for 30 to 40 minutes by using TFA:DCM (1:1) mixture to obtain free peptide amine (17);
(iii) coupling the free peptide amine (17) at the N-end with Boc-Ala-Ile-OH in the presence of HBTU/HOAt in DMF at a temperature from 0° C. to 40° C. to afford the Boc-protected pentapeptide (18);
(iv) subjecting the Boc-protected pentapeptide (18) to ester saponification at a temperature from 0° C. to 40° C. in water for 4 hours, followed by concurrent Boc-deprotection and O-demethylation by using HBr in AcOH at a temperature from 35° C. to 40° C. for 1 hour to afford deprotected pentapeptide (19).

In yet another embodiment, the present invention provides a novel peptide comprising:
(i) Tripeptide (15):

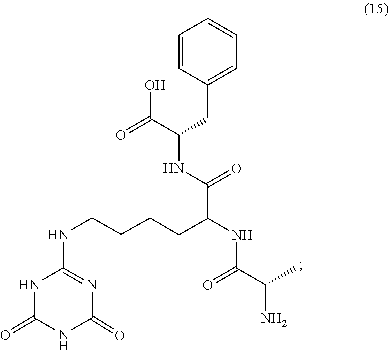 [SMILES: C[C@H](N)C(=O)NC(CCCCNc1nc(=O)[nH]c(=O)[nH]1)C(=O)N[C@@H](Cc1ccccc1)C(=O)O]

and
(ii) Pentapeptide (19);

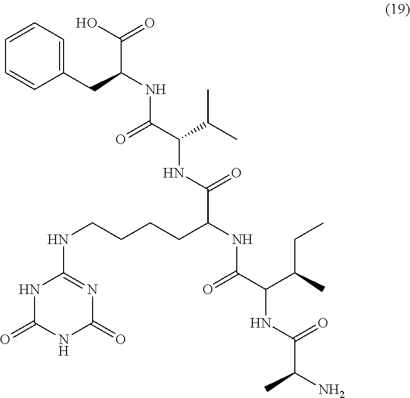 [SMILES: CC[C@@H](C)C(NC(=O)[C@H](C)N)C(=O)NC(CCCCNc1nc(=O)[nH]c(=O)[nH]1)C(=O)N[C@H](C(=O)N[C@@H](Cc1ccccc1)C(=O)O)C(C)C]

In yet another embodiment, the triple G-C-T nucleobase amino acids of Formula (11) and Formula (9) find wide-ranging application for nucleic acid recognition and nucleic acid-peptide/protein interaction studies.

### EXAMPLES

Unless otherwise stated, all chemicals and reagents were obtained commercially. Compound (10) was synthesized as per the reported procedure.

### Example 1

### General Procedures

### Boc-Deprotection of Compounds (12) and (16)

The Boc protected compounds (12) and (16) were subjected to deprotection by using TFA:DCM (1:1) mixture for 30 minutes at 0° C. to 40° C. After completion of reaction, the mixture was stripped off and co-evaporated with toluene:methanol (9:1) at least two times to afford the peptide amines (13) and (17), which were used for next steps without further purification.

### Hydrolysis of Esters (14) and (18) to Their Acids

To the solutions of esters (14) and (18) (1 equiv.) in methanol, LiOH·H₂O (5 equiv.) was added in water at 0° C. and the reaction mixture was stirred for 4 hours. After the complete consumption of the starting material, the solvent was evaporated under reduced pressure and the residue was treated with sat. KHSO₄ solution and was followed by extraction with EtOAc twice. The corresponding acid derivatives, obtained after evaporation of the solvent were taken for the next reaction without further purification.

### Demethylation and Boc Deprotection of (11), (14), and (18)

A solution of (11), (14), and (18) (1 equiv.) in 1 mL HBr in AcOH was stirred for 1 hour. Then, diethyl ether (Et₂O) was added to the reaction mixture and the resultant solid was washed three times with Et₂O and dried under vacuum giving (9), (15), and (19) respectively, which were hygroscopic.

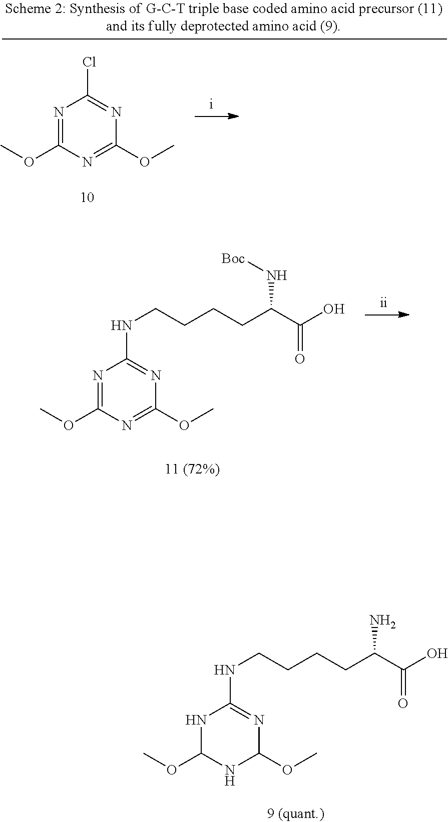

The reaction conditions for Scheme 2 are as follows:
(i) N_{α}-boc-L-lys-OH, DIPEA, MeOH, room temperature (rt), 3 h;
(ii) HBr in AcOH, rt, 1 h.

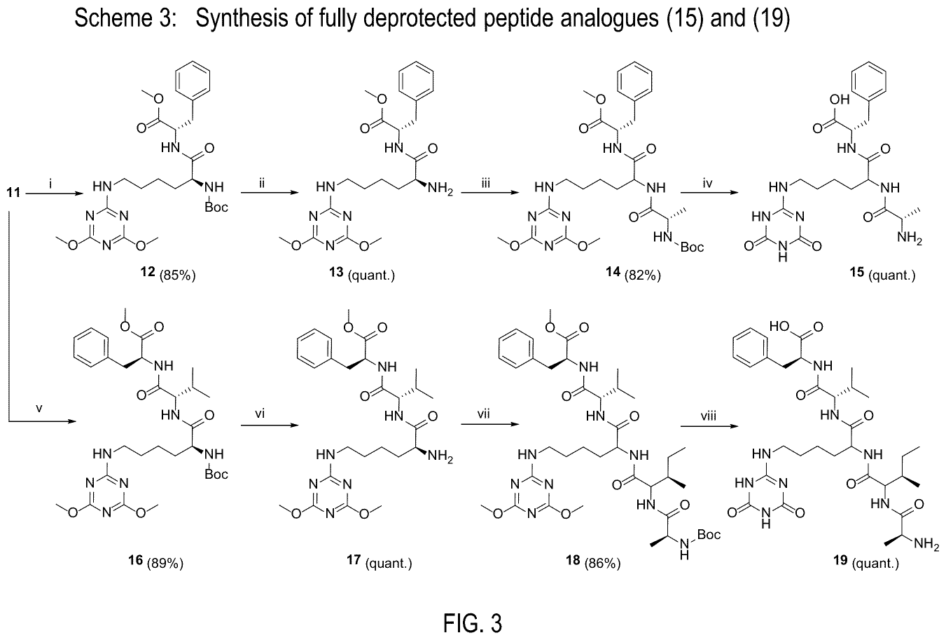

Scheme 3 for the synthesis of fully deprotected peptide analogues (15) and (19) is provided in FIG. 3. The reaction conditions Scheme 3 are as follows:
(i) NH₂-Phe-OMe. HCL salt, HBTU, HOAt, DIPEA, DMF, rt, 18 h;
(ii) TFA:DCM (1:1) 0° C. to rt, 0.5 h;
(iii) Boc-Ala-OH, HBTU, HOAt, DIPEA, DMF, rt, 18 h;
(iv) (a) LiOH, MeOH, H₂O, rt, 4 hrs; (b) HBr in AcOH, rt, 1 h;
(v) NH₂-Val-Phe-OMe, HBTU, HOAt, DIPEA, DMF, rt, 18 h;
(vi) TFA:DCM (1:1) 0° C. to rt, 0.5 h;
(vii) Boc-Ala-Ile-OH, HBTU, HOAt, DIPEA, DMF, rt, 18 h;
(viii) (a) LiOH, MeOH, H₂O, rt, 4 hrs; (b) HBr in AcOH, rt, 1 h.

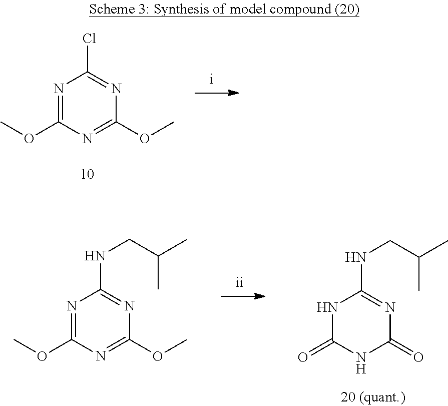

The reaction conditions for Scheme 3 are as follows:
(i) isobutyl amine, DIPEA, MeOH, rt, 3 h;
(ii) HBr in AcOH, rt, 1 h.

### Example 2

### Preparation of Compound (11)

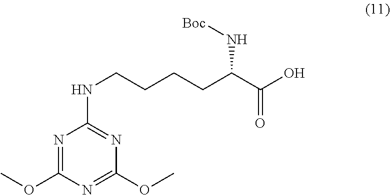 [SMILES: COc1nc(NCCCC[C@H](NC(=O)OC(C)(C)C)C(=O)O)nc(OC)n1]

Compound 10 (0.50 g, 2.857 mmol, 1 equiv.) was reacted with N_{α}-boc-L-lys-OH (0.913 g, 3.714 mmol, 1.3 equiv.) in dry methanol (8 mL) as solvent in the presence of N,N-diisopropylethylamine (DIPEA) (0.631 mL, 3.428 mmol, 1.2 equiv.) as a base at 35-40° C. for 2 hrs. After the completion of reaction, methanol was evaporated on rotavapor.

The resulting reaction mixture was dissolved in water, and the water layer was washed with diethyl ether. The aqueous layer further acidified with aq. KHSO₄ (pH=3-4), and product was extracted with EtOAc (3×25 mL). The organic layer was dried over Na₂SO₄ and concentrated in vacuum to afford the compound 11 (0.790 g, 71%) as a sticky liquid.

[α]²⁵_{D}=−1.63° (c=0.2, MeOH); ¹H NMR (400 MHz, CDCl₃) δ: 7.72 (bs, 1H), 5.45-5.43 (d, J=8.01 Hz, 1H), 4.39-4.36 (q, 1H), 3.97 (s, 3H), 3.93 (s, 3H), 3.61-3.57 (m, 1H), 3.29-3.27 (m, 1H), 1.88-1.81 (m, 2H), 1.65-1.54 (m, 2H), 1.44 (s, 9H), 1.44 (s, 1H), 1.28-1.24 (m, 1H); ¹³C NMR (100 MHz, CDCl₃) δ: 176.5, 173.4, 172.0, 171.1, 166.7, 155.3, 79.7, 55.3, 54.7, 54.7, 53.3, 40.6, 31.9, 28.5, 28.3, 21.8; HRMS (ESI) calculated [M+H]⁺ for C₁₆H₂₇N₅O₆: 385.1961, found 386.2039 [M+H]⁺.

### Example 3

### Preparation of Compound (9)

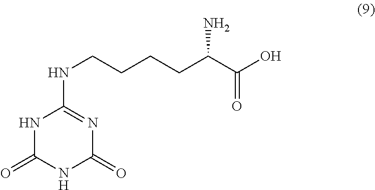 [SMILES: N[C@@H](CCCCNc1nc(=O)[nH]c(=O)[nH]1)C(=O)O]

The product (9) was obtained in quantitative yield as a hygroscopic solid using the general procedure for demethylation and Boc deprotection.

[α]²⁵_{D}=−0.13° (c=0.2, MeOH); ¹H NMR (500 MHz, D₂O) δ: 4.03-4.0 (t, 1H), 3.38-3.34 (t, 2H), 1.97-1.80 (m, 2H), 1.66-1.59 (m, 2H), 1.51-1.32 (m, 2H); ¹³C NMR (125 MHz, CDCl₃) δ: 171.8, 152.2, 150.7, 149.2, 52.6, 41.7, 29.2, 27.0, 21.3; HRMS (ESI) calculated [M+H]⁺ for C₉H₁₆N₅O₄: 257.1124, found 258.194 [M+H]⁺.

### Example 4

### Preparation of Compound (12)

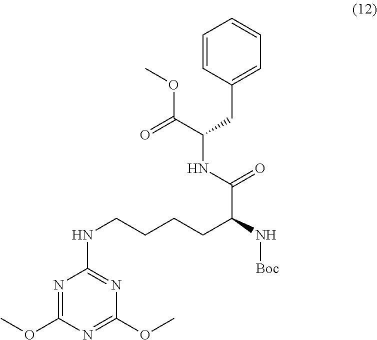 [SMILES: COC(=O)[C@H](Cc1ccccc1)NC(=O)[C@H](CCCCNc1nc(OC)nc(OC)n1)NC(=O)OC(C)(C)C]

A solution containing acid (11) (0.10 g, 0. 259 mmol, 1 equiv.) in dry DMF (2 mL) was cooled at 0° C. To this reaction mixture, HBTU (0.147 g, 0.389 mmol, 1.5 equiv.) was added followed by HOAt (0.028 g, 0.207 mmol, 0.8 equiv.) and DIPEA (0.167 mL, 0.909 mmol, 3.5 equiv.). Finally, L-phenylalanine methyl ester hydrochloride salt (0.072 g, 0.337 mmol, 1.3 equiv.) was added.

The reaction mixture was stirred at 0° C. for 10 minutes and then at 35-40° C. for 18 h. After completion of the reaction, reaction mixture was added into the ice water and extracted with EtOAc twice. The combined EtOAc layer was washed sequentially with sat. NaHCO₃, sat. KHSO₄, water and brine solution. EtOAc layer was then dried over Na₂SO₄, filtered, and concentrated under reduced pressure. Purification by column chromatography (eluent: 50% AcOEt/pet. ether, Rf: 0.5) afforded compound (12) (0.120 g, 85%) as a white solid. mp: 87-89° C.;

[α]²⁵_{D}=−8.07° (c=0.2, MeOH); ¹H NMR (400 MHz, CDCl₃) δ: 7.29-7.21 (m, 3H), 7.11-7.09 (m, 2H), 6.58-6.56 (d, J=7.38 Hz, 1H), 5.72 (s, 1H), 5.05-5.03 (d, J=7.13 Hz, 1H), 4.87-4.82 (q, 1H), 4.12-4.06 (m, 1H), 3.94 (s, 3H), 3.90 (s, 3H), 3.71 (s, 3H), 3.42-3.37 (q, 2H), 3.17-3.05 (m, 2H), 1.84-1.75(m, 1H), 1.64-1.50 (m, 3H), 1.42 (s, 9H), 1.39-1.33 (m, 2H); ¹³C NMR (100 MHz, CDCl₃) δ: 172.4, 171.8, 177.7, 177.6, 168.0, 155.4, 135.6, 129.1, 128.5, 127.0, 80.0, 54.5, 54.4, 54.2, 53.0, 52.3, 40.5, 37.7, 32.0, 28.8, 28.2, 22.5; HRMS (ESI) calculated [M+H]⁺ for C₂₆H₃₉N₆O₇: 546.2802, found 547.2883 [M+H]⁺.

### Example 5

### Preparation of Compound (14)

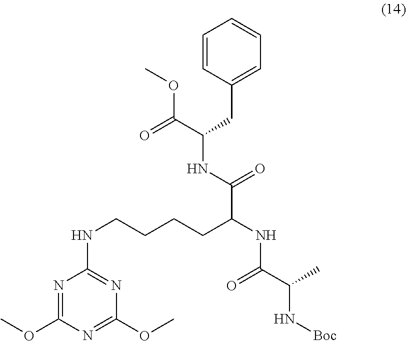 [SMILES: COC(=O)[C@H](Cc1ccccc1)NC(=O)C(CCCCNc1nc(OC)nc(OC)n1)NC(=O)[C@H](C)NC(=O)OC(C)(C)C]

A solution Boc-ala-OH (0.100 g, 0. 460 mmol, 1 equiv.) in dry DMF (3 mL) was cooled at 0° C. To the reaction mixture HBTU (0.261 g, 0.691 mmol, 1.5 equiv.), HOAt (0.050 g, 0.368 mmol, 0.8 equiv.) and DIPEA (0.297 mL, 1.612 mmol, 3.5 equiv.) were added. Finally, free amine (13) (0.267 g, 0.599 mmol, 1.3 equiv.) was added.

The reaction mixture was stirred at 0° C. for 10 minutes and at 35-40° C. for 18 h. After completion of the reaction, reaction mixture was added into ice water and extracted with EtOAc twice. The combined EtOAc layer was washed sequentially with sat. NaHCO₃, sat. KHSO₄, water and brine solution. EtOAc layer was then dried over Na₂SO₄, filtered, and concentrated under reduced pressure. Purification was by column chromatography (eluent: 80% AcOEt/pet. ether, Rf: 0.5) afforded (14) (0.245 g, 82%) as a white solid. mp: 143-145° C.

[α]²⁵_{D}=−25.01° (c=0.2, MeOH); ¹H NMR (500 MHz, CDCl₃) δ: 7.25-7.17 (m, 3H), 7.11 (bs, 1H), 7.08-7.07 (m, 2H), 6.95 (bs, 1H), 6.32 (bs, 1H), 5.56 (bs, 1H), 4.82-4.78 (q, 1H), 4.45-4.41 (q, 1H), 4.23 (bs, 1H), 3.91 (s, 3H), 3.88 (s, 3H), 3.68 (s, 3H), 3.37-3.33 (q, 2H), 3.12-3.02 (m, 2H),1.81-1.74(m, 1H), 1.63-1.47 (m, 3H), 1.38 (s, 9H), 1.34-1.27 (m, 6H); ¹³C NMR (125 MHz, CDCl₃) δ: 173.1, 172.3, 171.7, 171.1, 167.8, 155.5, 135.7, 129.0, 128.4, 126.9, 79.8, 54.4, 54.3, 53.2, 52.7, 52.2, 49.8, 40.4, 37.5, 31.7, 29.5, 28.4, 28.1, 22.3, 18.0; HRMS (ESI) calculated [M+H]⁺ for C₂₉H₄₃N₇O₈: 617.3173, found 618.3252 [M+H]⁺.

### Example 6

### Preparation of Compound (15)

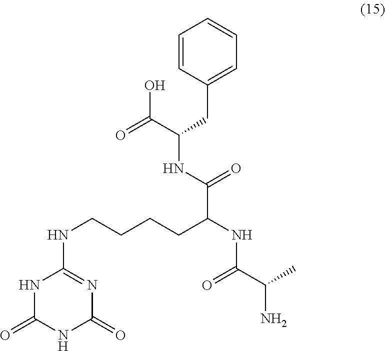 [SMILES: C[C@H](N)C(=O)NC(CCCCNc1nc(=O)[nH]c(=O)[nH]1)C(=O)N[C@@H](Cc1ccccc1)C(=O)O]

The compound (15) was obtained in quantitative yield as a hygroscopic solid using the general procedure for demethylation and Boc deprotection. [α]²⁵_{D}=−0.15° (c=0.2, MeOH); ¹H NMR (500 MHz, D₂O) δ: 7.23-7.21 (m, 2H), 7.17-7.13 (m, 3H), 4.56-4.54 (m, 1H), 4.15-4.13 (t, 1H), 3.99-3.95 (m, 1H), 3.27-3.21 (m, 2H), 3.10-3.07 (m, 1H), 2.94-2.85 (m, 1H), 1.58-1.55 (m, 2H), 1.52-1.49 (m, 2H), 1.34-1.31 (d, 3H), 1.27-1.13 (m, 2H); ¹³C NMR (125 MHz, D₂O) δ: 174.3, 173.0, 170.4, 151.9, 150.3, 149.0, 136.3, 129.2, 128.6, 127.0,53.7, 53.7, 48.8, 41.9, 36.5, 30.6, 27.0, 21.9, 16.6; HRMS (ESI) calculated [M+H]⁺ for C₂₁H₃₀N₇O₆: 475.2179, found 476.2255 [M+H]⁺.

### Example 7

### Preparation of Compound (16)

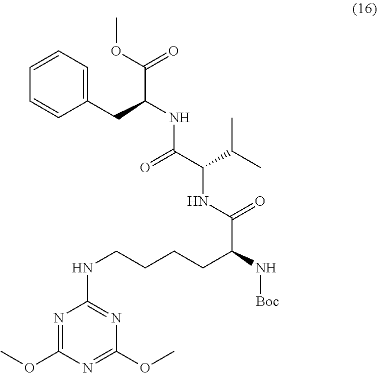 [SMILES: COC(=O)[C@H](Cc1ccccc1)NC(=O)[C@@H](NC(=O)[C@H](CCCCNc1nc(OC)nc(OC)n1)NC(=O)OC(C)(C)C)C(C)C]

A solution containing acid (11) (0.100 g, 0.259 mmol, 1 equiv.) in dry DMF (2 mL) was cooled at 0° C. To the reaction mixture was added HBTU (0.147 g, 0.389 mmol, 1.5 equiv.) followed by HOAt (0.028 g, 0.207 mmol, 0.8 equiv.) and DIPEA (0.167 mL, 0.909 mmol, 3.5 equiv.). Finally, the dipeptide (NH₂-Val-Phe-OMe) (0.072 g, 0.337 mmol, 1.3 equiv.) was added.

The reaction mixture was stirred at 0° C. for 10 minutes and at 35-40° C. for 18 h. After completion of the reaction, reaction mixture was added into the ice water. The resulting solid was filtered and dried under vacuum affording (16) (89%) as a white solid. Note: Before submitting for NMR, compound (16) was washed with Et₂O twice. mp: 150-152° C.;

[α]²⁵_{D}=−23.82° (c=0.2, MeOH); ¹H NMR (400 MHz, CDCl₃) δ: 7.31-7.22 (m, 3H), 7.11-7.09 (m, 2H), 6.76-6.74 (d, J=8.25 Hz, 1H), 6.57-6.55 (d, J=7.50 Hz, 1H), 5.82 (s, 1H), 5.14-5.12 (d, J=7.88 Hz, 1H), 4.90-4.85 (q, 1H), 4.28-4.24 (m, 1H), 4.08-4.05 (m, 1H), 3.95 (s, 3H), 3.91 (s, 3H), 3.71 (s, 3H), 3.44-3.39 (q, 2H), 3.11-3.09 (m, 2H), 1.87-1.78(m, 1H), 1.68-1.53 (m, 3H), 1.43 (s, 9H), 1.40-1.37 (m, 2H), 0.92-0.88 (m, 6H); ¹³C NMR (100 MHz, CDCl₃) δ: 172.4, 172.1, 171.9, 171.7, 170.6, 168.0, 135.6, 129.1, 128.5, 127.1, 80.1, 58.4, 54.5, 54.4, 53.1, 52.3, 40.5, 37.8, 30.7, 29.6, 28.8, 28.2, 22.7, 19.0, 17.7; HRMS (ESI) calculated [M+H]⁺ for C₃₁H₄₈N₇O₈: 645.3486, found 646.3564 [M+H]⁺.

### Example 8

### Preparation of Compound (18)

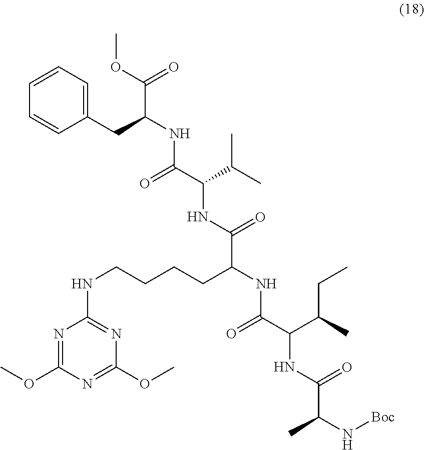 [SMILES: CC[C@@H](C)C(NC(=O)[C@H](C)NC(=O)OC(C)(C)C)C(=O)NC(CCCCNc1nc(OC)nc(OC)n1)C(=O)N[C@H](C(=O)N[C@@H](Cc1ccccc1)C(=O)OC)C(C)C]

A solution Boc-Ala-Ile-OH (0.050 g, 0.165 mmol, 1 equiv.) in dry DMF (2 mL) was cooled to 0° C. To the reaction was added HBTU (0.094 g, 0.248 mmol, 1.5 equiv.) followed by HOAt (0.018 g, 0.132 mmol, 0.8 equiv.) and DIPEA (0.106 mL, 0.579 mmol, 3.5 equiv.). Finally, free amine (17) (0.117 g, 0.215 mmol, 1.3 equiv.) was added.

The reaction mixture was stirred at 0° C. for 10 minutes and at 35-40° C. for 18 h. After completion of the reaction, reaction mixture was added into ice water. The resulting solid was filtered and dried under vacuum affording (10) (85%) as a white solid. Note: Before submitting for NMR, compound 18 was washed with Et₂O twice. mp: 226-228° C.;

[α]²⁵_{D}=143.44° (c=0.2, MeOH); ¹H NMR (400 MHz, DMSo-d₆) δ: 8.40-8.37 (t, 1H)ᵣₒₜₐₘₑᵣ, 8.06-7.98 (m, 1H)ᵣₒₜₐ, 7.87-7.84 (m, 1H)ᵣₒₜₐₘₑᵣ, 7.72-7.65 (dd, 1H)ᵣₒₜₐₘₑᵣ, 7.60-7.55 (dd, 1H)ᵣₒₜₐₘₑᵣ, 7.26-7.23 (m, 2H), 7.20-7.17 (m, 3H), 7.04-6.94 (dd, 1H)ᵣₒₜₐₘₑᵣ, 4.49-4.44 (m, 1H)ᵣₒₜₐₘₑᵣ, 4.37-4.35 (m, 1H)ᵣₒₜₐₘₑᵣ, 4.28-4.24 (m, 1H)ᵣₒₜₐₘₑᵣ, 4.21-4.14 (m, 1H)ᵣₒₜₐₘₑᵣ, 4.04-3.93 (m, 1H)ᵣₒₜₐₘₑᵣ, 3.80 (s, 3H), 3.79 (s, 3H), 3.55 (s, 3H), 3.24-3.18 (m, 2H)ᵣₒₜₐₘₑᵣ, 3.03-2.99 (m, 2H)ᵣₒₜₐₘₑᵣ, 2.94-2.89 (m, 1H)ᵣₒₜₐₘₑᵣ, 1.93-1.85 (m, 1H)ᵣₒₜₐₘₑᵣ, 1.75-1.67 (m, 1H)ᵣₒₜₐₘₑᵣ, 1.57-1.55 (m, 1H)ᵣₒₜₐₘₑᵣ, 1.48-1.42 (m, 3H)ᵣₒₜₐₘₑᵣ, 1.36 (s, 9H), 1.27-1.21 (m, 3H)ᵣₒₜₐₘₑᵣ, 1.16-1.13 (t, 3H)ᵣₒₜₐₘₑᵣ, 1.07-1.01 (m, 1H)ᵣₒₜₐₘₑᵣ, 0.82-0.72 (m, 12H)ᵣₒₜₐₘₑᵣ; ¹³C NMR (100 MHz, CDCl₃) δ: 172.7, 172.5, 172.0, 171.7, 171.5,171.4, 171.2, 171.0, 171.0, 171.0, 170.9, 170.7, 167.6, 167.5, 155.1, 155.0, 137.0, 128.9, 128.9, 128.2, 126.5, 79.1, 78.1, 57.2, 57.1, 56.5, 55.2, 54.0, 54.0, 53.9, 53.4, 52.5, 52.3, 51.7, 50.1, 49.8, 37.3, 37.0, 36.5, 31.6, 31.3, 30.9, 30.7, 28.4, 28.1, 25.7, 24.0, 22.7, 22.7, 19.0, 18.9, 18.2, 17.9, 17.9, 17.8, 15.2, 14.1, 11.4, 11.0; HRMS (ESI) calculated [M+H]⁺ for C₄₀H₆₃N₉O₁₀: 829.4698, found 830.4776 [M+H]⁺.

### Example 9

### Preparation of Compound (19)

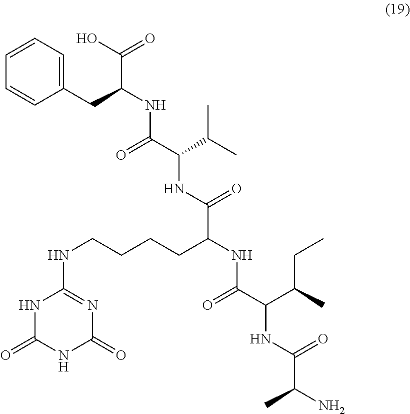 [SMILES: CC[C@@H](C)C(NC(=O)[C@H](C)N)C(=O)NC(CCCCNc1nc(=O)[nH]c(=O)[nH]1)C(=O)N[C@H](C(=O)N[C@@H](Cc1ccccc1)C(=O)O)C(C)C]

The product (19) was obtained in quantitative yield as a hygroscopic solid using the general procedure for demethylation and Boc deprotection. [α]²⁵_{D}=−0.19° (c=0.2, MeOH); ¹H NMR (400 MHz, CDCl₃) δ: 7.26-7.21 (m, 3H), 7.17-7.15 (m, 2H), 4.62-4.58 (m, 2H), 4.27-4.21 (m, 2H), 4.13-4.00 (m, 3H), 3.36-3.11 (m, 1H), 2.98-2.92 (m, 1H), 1.95-1.81 (m, 2H), 1.79-1.71 (m, 1H), 1.66-1.63 (m, 2H), 1.61-1.54 (m, 2H), 1.49-1.44 (m, 4H), 1.33-1.20 (m, 3H), 1-18-1.09 (m, 1H), 0.84-0.78 (m, 12H); ¹³C NMR (100 MHz, CDCl₃) δ: 174.2, 173.3, 173.1, 172.8, 172.7, 171.1, 170.6, 151.3, 149.2, 149.1, 148.4,136.4, 129.1, 128.6, 127.0, 59.1, 58.3, 57.6, 53.8, 53.5, 53.2, 49.1, 48.8, 42.2, 36.5, 36.3, 36.1, 30.5, 30.4, 30.3, 30.1, 26.9, 25.5, 24.5, 22.2, 22.0, 18.2, 17.8, 17.7, 16.8, 16.7, 14.6, 14.0, 10.6, 10.2; HRMS (ESI) calculated [M+H]⁺ for C₃₂H₅₀N₉O₈: 687.3704, found 688.3778 [M+H]⁺.

### Example 10

### Preparation of Compound (20)

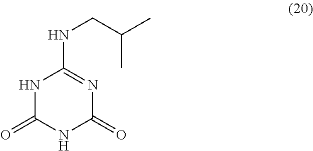

The intermediate N-isobutyl-4,6-dimethoxy-1,3,5-triazin-2-amine was synthesized by using the same procedure for compound (11) by using isobutyl amine. Finally, the compound was obtained in quantitative yield as a white solid using the general procedure for demethylation and Boc deprotection. ¹H NMR (400 MHz, DMSO-d₆) δ: 11.65 (bs, 1H), 11.15 (s, 1H), 8.73 (bs, 1H), 3.17-3.16 (d, J=6. 87 Hz, 2H), 1.85-1.77 (bs, 1H), 0.88-0.87 (d, J=6. 87 Hz, 6H); ¹³C NMR (100 MHz, DMSO-d₆) δ: 152.7, 150.1, 150.0, 148.1, 48.2, 27.6, 19.6.

### Example 11

### X-Ray Crystal Structure Analysis of Compound (20)

About 0.1 g of 20 was dissolved in 1.5 mL of dimethylformamide. This solution was kept at 35-40° C. for 4 days and afforded needle-shaped colorless crystals.

Single-crystal data of compound 20 was collected on a Bruker SMART APEX four-circle diffractometer equipped with a CMOS photon 100 detector (Bruker Systems Inc.) and with a Cu Kα radiation (1.5418 Å). The incident X-ray was focused and monochromated using Micro focus (IμS). Crystal of compound 20 was mounted on nylon Cryo loops with Paratone-N oil. Full data was collected at 100 K maintained by “Cryoconnector” and liquid nitrogen. Data was integrated using Bruker SAINT software and was corrected for absorption using SADABS. Structure was solved by Intrinsic Phasing method and refined using the SHELXTL 2017 software suite. All non-hydrogen atoms were located from iterative examination of difference F-maps, following which, the structure was refined using a least-squares method. Hydrogen atoms were placed geometrically and placed in a riding model. The parameters are given in TABLE 1 below.

TABLE 1

| Parameters | Compound 12 | |
|---|---|---|
| Chemical formula | C₇H₁₂N₄O₂ | |
| Formula weight | 184.19 | |
| Crystal system | Triclinic | |
| Space group | P-1 | |
| Unit-cell parameters | a = 9.132 (7) Å | α = 76.88 (8)° |
| | b = 9.455 (11) Å | β = 89.63(7)° |
| | c = 14.002(15) Å | γ = 71.51(6)° |

| | |
|---|---|
| Crystal color and shape | Colorless-needle |
| Temperature | 100K |
| No. of formula units in | 2 |
| the unit cell (Z) | |
| Density (g cm⁻¹) | 1.32 |
| Abs. Coeff. (mm⁻¹) | 0.831 |
| F(000) | 472 |
| Reflection data | |
| No. of reflections meas. | 19754 |
| No. of unique reflections | 3963 |
| No. of obs. reflections | 2737 |
| λ (Å) | 1.54178 |
| R_{merge} | 0.092 |
| Av. I/sig(1) | 14.90 |
| Index ranges | −10 ≤ h ≤ 9, |
| | −11 ≤ k ≤ 11, |
| | −16 ≤ l ≤ 16 |
| θₘₐₓ | 68.7 |
| θₘᵢₙ | 5.1 |
| Refinement Data | |
| Absorption correction type | multi-scan |
| Tₘᵢₙ | 0.638 |
| Tₘₐₓ | 0.779 |
| Rₐₗₗ | 0.126 |
| R_{obs} | 0.092 |
| wR₂(all) | 0.281 |
| wR₂(obs) | 0.259 |
| Goodness-of-fit (GOOF) | 1.070 |
| Largest diff. peak and hole: | 0.524 |
| Delta-rho (eÅ⁻³)ₘₐₓ | |
| Largest diff. peak and hole: | −0.369 |
| Delta-rho (eÅ⁻³)ₘᵢₙ | |

Thus, the present disclosure provides a triple G-C-T nucleobase amino acid (G-C-TNBA), featuring three recognition faces: DDA (G mimic), DAA (C mimic) and ADA (T mimic). The G-C-TNBA is readily obtainable in multi-gram scale in a remarkably facile one-step reaction.

Owing to the prototropy effect, the triple G-C-T base can exist in two forms, displaying G and T faces and C and T faces. This suggests that it can simultaneously interact with two sets of nucleobases (C-A or G-A) at any given time.

We claim:

1. A triple G-C-T base coded nucleobase amino acid, comprising a compound according to formula (I): 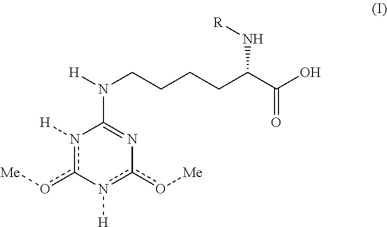
where R is —H or -Boc.
wherein:
the triple G-C-T base coded nucleobase amino acid comprises three recognition sites that can simultaneously interact with two sets of nucleobases at any given time;
the three recognition sites comprise DDA as a G mimic, DAA as a C mimic, and ADA as a T mimic; and
the two sets of nucleobases comprise C-A and G-A.

2. The triple G-C-T base coded nucleobase amino acid of claim 1, wherein the compound according to formula (I) is compound (9) or compound (11):

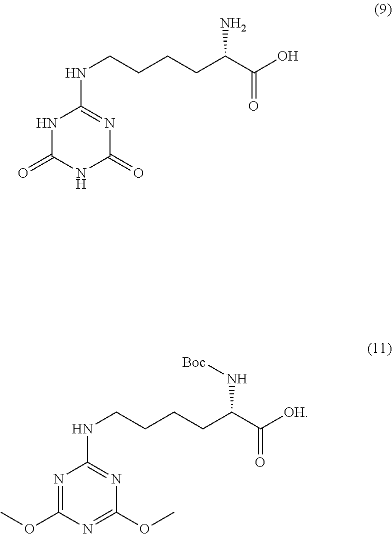 [SMILES: N[C@@H](CCCCNc1nc(=O)[nH]c(=O)[nH]1)C(=O)O ; COc1nc(NCCCC[C@H](NC(=O)OC(C)(C)C)C(=O)O)nc(OC)n1]

3. The triple G-C-T base coded nucleobase amino acid of claim 2, wherein the compound according to formula (I) is compound (9).

4. The triple G-C-T base coded nucleobase amino acid of claim 2, wherein the compound according to formula (I) is compound (11).

5. A method for preparing the triple G-C-T amino acid according to claim 3, the method comprising:
(a) reacting 2-chloro-4,6-dimethoxy-1,3,5-triazine (10): 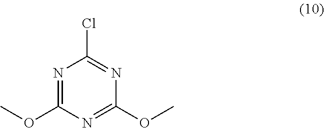
with α-Boc lysine in the presence of base and solvent at a temperature from 35° C. to 40° C. for 1 hour to 2 hours to obtain a Boc-protected G-C-T-amino acid intermediate (11): 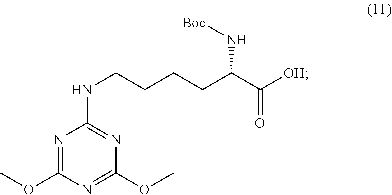 [SMILES: COc1nc(NCCCC[C@H](NC(=O)OC(C)(C)C)C(=O)O)nc(OC)n1]
and
(b) deprotecting the Boc-protected G-C-T-amino acid intermediate (11) with HBr/AcOH at a temperature from 35° C. to 40° C. for 1 hour to remove a Boc group and demethylate, to obtain, in quantitative yield, free triple G-C-T amino acid compound (9):

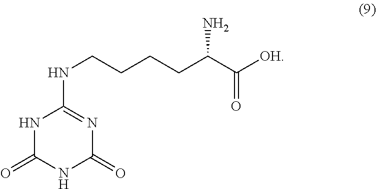 [SMILES: N[C@@H](CCCCNc1nc(=O)[nH]c(=O)[nH]1)C(=O)O]

6. The method according to claim 5, wherein the base is N,N-diisopropylethyl amine and the solvent is dry methanol.

7. A method for preparing the triple G-C-T amino acid according to claim 4, the method comprising:
reacting 2-chloro-4,6-dimethoxy-1,3,5-triazine (10): 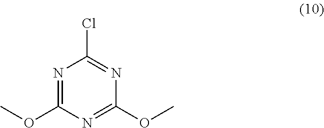
with α-Boc lysine in the presence of base and solvent at a temperature from 35° C. to 40° C. for 1 hour to 2 hours to obtain compound (11):

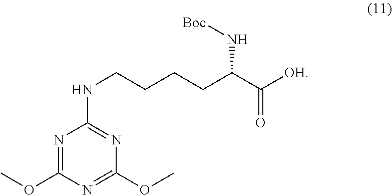 [SMILES: COc1nc(NCCCC[C@H](NC(=O)OC(C)(C)C)C(=O)O)nc(OC)n1]

8. The method according to claim 7, wherein the base is N,N-diisopropylethyl amine and the solvent is dry methanol.

9. A peptide synthesized from the triple G-C-T base coded nucleobase amino acid according to claim 4 as a key NBA building block intermediate, the peptide being chosen from:
a tripeptide compound (15): 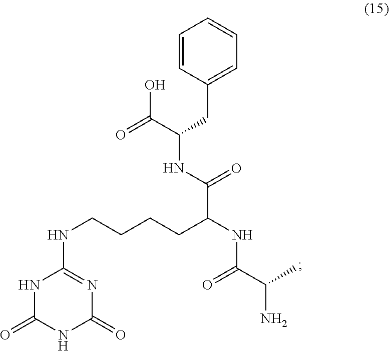 [SMILES: C[C@H](N)C(=O)NC(CCCCNc1nc(=O)[nH]c(=O)[nH]1)C(=O)N[C@@H](Cc1ccccc1)C(=O)O]
or
a pentapeptide compound (19):

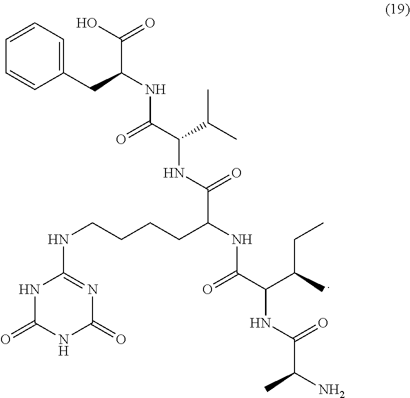 [SMILES: CC[C@@H](C)C(NC(=O)[C@H](C)N)C(=O)NC(CCCCNc1nc(=O)[nH]c(=O)[nH]1)C(=O)N[C@H](C(=O)N[C@@H](Cc1ccccc1)C(=O)O)C(C)C]

10. A method for synthesizing the peptide according to claim 9, the method comprising:
coupling C-termini of compound (11) with an alpha-amino acid in the presence of HBTU/HOAt in DMF at a temperature from 0° C. to 40° C. for 17 to 18 hours to obtain a peptide;
deprotecting a Boc group at a temperature from 0° C. to 40° C. for 30 to 40 minutes with a TFA:DCM (1:1) mixture to obtain a free peptide amine;
coupling the free peptide amine at an N-terminus further with Boc-protected alpha amino acid in the presence of HBTU/HOAt in DMF at a temperature from 0° C. to 40° C. for 17 to 18 hours to obtain a Boc-protected peptide sequence; and
saponifying, deprotecting, and O-demethylating the Boc-protected peptide sequence to obtain the tripeptide compound (15) or the pentapeptide compound (19).
